(19) United States
(12) Patent Application Publication
(10) Pub. No.: US 2012/0310172 A1
(43) Pub. Date: Dec. 6, 2012

(54) Drug Delivery Device

(75) Inventors: Catherine Anne MacDonald, Ashby-de-la-Zouch (GB); Robert Veasey, Leamington Spa (GB); Garen Kouyoumjian, Leamington Spa (GB); Christopher Jones, Tewkesbury (GB); Andrew Mark Lindsay, Hinckley (GB); Michael Bainton, Kineton (GB)

(73) Assignee: SANOFI-AVENTIS DEUTSCHLAND GMBH, Frankfurt am Main (DE)

(21) Appl. No.: 13/498,610

(22) PCT Filed: Sep. 29, 2010
(86) PCT No.: PCT/EP2010/064403

§ 371 Date: Aug. 3, 2012

(30) Foreign Application Priority Data
Sep. 30, 2009 (EP) 09171745.4

(51) Int. Cl. A61M 5/31

(52) U.S. Cl. 604/207

(57) ABSTRACT

A drug delivery device (10) is given, comprising a body (12) and a moveable button member (14), wherein in a pre-ready state of the drug delivery device (10) there is a misalignment between the body (12) and the button member (14). This misalignment is perceivable by a user of the drug delivery device (10). In a ready state of the drug delivery device (10), the button member (14) and the body (12) are aligned and the alignment is perceivable by a user of the drug delivery device (10).

The present disclosure relates to a drug delivery device.

Drug delivery devices are generally known for the administration of a medicinal product, like for example heparin, insulin or human growth hormones. The medicinal product may be self administered by a patient.

Before the first use of the drug delivery device, the patient usually has to prepare the drug delivery device for later dispense of a medicinal product. Users who are unfamiliar with such a prepareable drug delivery device may fail or incorrectly prepare the device before dispensing the first dose.

It is an object of the present disclosure to provide a drug delivery device, which helps to improve the accuracy of the first dispensed dose of a medicinal product and which makes the administration of a medicinal product safer.

The term “drug delivery device” may imply a device of any shape, for example the device might be pen-shaped. The device may deliver a single dose or multiple doses of a medicinal product. The dose can be pre-set, pre-defined or selectable. The drug delivery device can be disposable or reusable. Furthermore, the said drug delivery device may comprise a needle or may be needle-free. An attached needle can be fixed or replaceable.

The term “medicinal product” or “drug”, as used herein, preferably means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)₅-des Pro36, des Pro37 Exendin-4(1-39)-NH2,

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative;
or an Exendin-4 derivative of the sequence

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2,

### des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38 [Met(O)14, Trp(O2)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(O2)25, Asp28] Exendin-4(1-39)-NH2,

### des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(S1-39)-(Lys)6-NH2,

### H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

The term “body” may imply the external structure like for example a main body portion or an outer shell of the drug delivery device. The body can be a part of a housing, which might comprise a cartridge holder.

The term “cartridge holder” may imply an attachable or rigid part of the drug delivery device. Inside the cartridge holder, a medicament cartridge may be located which may contain for example a medicinal product. The cartridge holder may be attached to the body of the drug delivery device, thereby forming part of the housing.

The body and the cartridge holder may form the housing of the drug delivery device and may be designed to enable a safe, correct and comfortable handling of the drug delivery device. Usually it is designed to house, protect and guide other parts during operation of the device. It is also possible that the body engages with inner components of the drug delivery device such as for example the drive mechanism, the medicament cartridge and the piston rod.

The body is limiting the exposure to contaminants, such as liquid, dirt or dust. The cartridge holder may serve to house a medicament cartridge which might be replaceable or non-replaceable. A number of doses of a liquid medicinal product may be dispensed out of an assembled medicament cartridge.

The term “button member” may imply an element which might be located at the proximal end of the drug delivery device. The user may operate the button member to operate the drug delivery device. The button member may be moveable relative to the body of the drug delivery device. Moveable may for example include axial movement, rotational movement and other twisting movements.

The term “pre-ready state” may imply the state in which the drug delivery device is before the first use. If the drug delivery device is in the pre-ready state, the user usually has to prepare the device before the user is enabled to set and dispense a dose of a medicinal product. The preparing makes the drug delivery device ready for use. In the pre-ready state, the button member may be rotatable relative to the body.

The term “preparation” may imply to make the drug delivery device ready or suitable in advance for a particular purpose or for use. The preparation may include the priming of the drug delivery device. Thereby, the parts of the drug delivery device are moved to a position such that the drug delivery is ready for use after preparing the drug delivery device. As the medicament cartridge may comprise two different medicinal product components, preparation of the drug delivery device may include mixing the two components. After preparing the device, a dose of a medicinal product can be set and dispensed. Preparing also includes decreasing any tolerance gaps between mechanical parts of the drug delivery device.

Before preparing the drug delivery device, the button member can be lightly held in position by a detent feature. The detent feature prevents undesired movement like for example rattling of the mechanism before use. The detent feature can be for example located between the body and the button member.

The preparation of the drug delivery device is useful for the dose accuracy and for flushing a needle which might be fitted to an assembled medicament cartridge. Therefore, it is useful to draw the attention of the user to the need to prepare the drug delivery device. It may also be useful to encourage the user to prepare the drug delivery device through a recognizably different action before dose setting and dose dispensing actions are enabled.

The term “misalignment” may imply that components of the drug delivery device are not properly aligned. In the present disclosure, the components that are misaligned with respect to each other may be the button member and the body of the drug delivery device. Both, the button member and the body, are bigger sized components that are located at the surface of the drug delivery device and which are accessible and visible for the user.

The misalignment might be at the proximal end of the drug delivery device because a misalignment at this position together with the size of the button member and the body attracts the attention of the user. A cross section along a lateral axis of the body and the button member may be non-circular and non-rotation-symmetrically constructed.

A safety feature, like for example a detent, may avoid accidental or partial preparing by holding the relative position between the body and the button member.

The term “perceivable” may imply that the misalignment may be at least one of audibly, tactile or visibly indicated to the user of the drug delivery device. To mention just a few examples, the misalignment can be perceivable by the colour, the texture or the shape of the drug delivery device.

The term “ready state” may imply that the drug delivery device is in a state, in which a dose of a medicinal product may be set and dispensed. The drug delivery device may get into the ready state simultaneously with the alignment of the button member and the body.

By rotation of the button member for example by 90° relative to the body, the drug delivery device gets from the pre-ready state to the ready state. After the drug delivery device is prepared, the button member may be no longer rotatable with respect to the body of the drug delivery device.

In another embodiment, the button member can be substantially rotated by the user but have an additional axial component of movement. This movement can for example be along a helical path until the preparation step has been completed. After the drug delivery device has reached the ready state, axial motion may be permitted for dose setting and dispensing.

In the ready state, the button member is axially displaceable with respect to the body. The preparation procedure of the drug delivery device might be an irreversible action. After having reached the ready state, the drug delivery device may be irreversibly kept in the ready state. At the end of the twist action, a lug that might be located inside the drug delivery device snaps over a clip on the button member so that the user can no longer rotate the button.

In the ready state, the button member may be axially moveable relative to the body to set and dispense a dose of a medicinal product.

The term “transient state” may imply that the drug delivery device is in a state in which the drug delivery device is changing from the pre-ready state to the ready state. The transient state switches over to the ready state as soon as the button member is aligned with the body of the drug delivery device. To get from the pre-ready state to the ready state, the user might have to rotationally twist the body and the button member relative to the body.

The term “drive assembly” may comprise at least one of for example a drive member, a piston rod, a drive sleeve and a lead screw. The drug delivery device may be electronically or mechanically operated to expel a medicinal product from the drug delivery device.

The drive sleeve may be operationally coupled with the button member and may be located inside the body of the drug delivery device. By operating the button member the user may prepare the drug delivery device to allow a dose of a medicinal product to be set and dispensed. The drive member may comprise a first portion, which is rigidly attached to the body and a second portion, which is moveable with respect to the body.

The term “window” may imply a transparent opening that allows vision through which additional information are visible. Windows can be located for example on the body of the drug delivery device through which indicators located on the drive sleeve or button can be seen, and also on the cartridge holder to check the liquid level of a medicament cartridge.

The term “indicator” may imply a signal for attracting the attention of a user of the drug delivery device. The at least one indicator, like for example arrows or other symbols, might be located on the drive sleeve or button, and can be seen through a window in the body of the drug delivery device.

There might be different indicators to indicate the different states of the drug delivery device and to indicate the possible directions of movement of the button member relative to the body. The pre-ready, the transient or the ready status can be indicated by the indicators on the drive sleeve or button. The movements to set and dispense a dose of a medicinal product can also be indicated.

It is desirable that, for example, a detent feature keeps the button member in the pre-ready state so that any indicators are appropriately oriented for the user to see for example arrows which are marked on the drive sleeve or button and visible through a window in the body of the drug delivery device. The orientation of the arrow in relation to the drug delivery device indicates the next step for preparing the drug delivery device or the next step for setting and dispensing a dose of a medicinal product. For example, when the arrow points in a direction perpendicular to the axis of the device, the user should twist the button in that direction relative to the body of the drug delivery device.

The term “pattern” may imply that it comprises at least one of a haptic feature, a coloured line, a symbol, a haptic symbol and a coloured symbol. One part of the pattern might be on the body and the complementary on the button member. These two pattern-parts match together and complete each other.

This completion of the pattern is perceivable by the user. In a pre-ready state, the preparation status is detectable by a misalignment of a pattern shown on both the body and the button member. The ready state of the drug delivery device is detectable by an alignment of the pattern on the body and the button member. In other words, the ready status is easily visible and/or additionally haptically perceivable to the user without a closer look at the drug delivery device.

The term “detectable” may imply something that is perceivable by the user or by an adapted sensor.

The term “sensor” may imply any device that receives a signal or stimulus as temperature change or pressure or light or motion and so on and responds to it. The sensor might be a distance sensor or a limit switch or a sensor which closes a circuit if the button member and the body are aligned and prepared. The sensor might be on any part of the drug delivery device and might detect the preparation status.

The term “display” may imply any electronic device that represents information in visual form. For example a liquid crystal display. The display may indicate the preparation status visibly for the user of the drug delivery device. For example the display can indicate that the drug delivery device has reached the ready state or is in the ready state. But also any other information may be displayed.

A drug delivery device may comprise a body and a moveable button member. In a pre-ready state of the drug delivery device, there might be a misalignment between the body and the button member which is perceivable by a user of the drug delivery device. In a ready state of the drug delivery device, the button member and the body are aligned and the alignment is perceivable by a user of the drug delivery device.

In the following, the disclosure is described in further detail with reference to the drawings, wherein

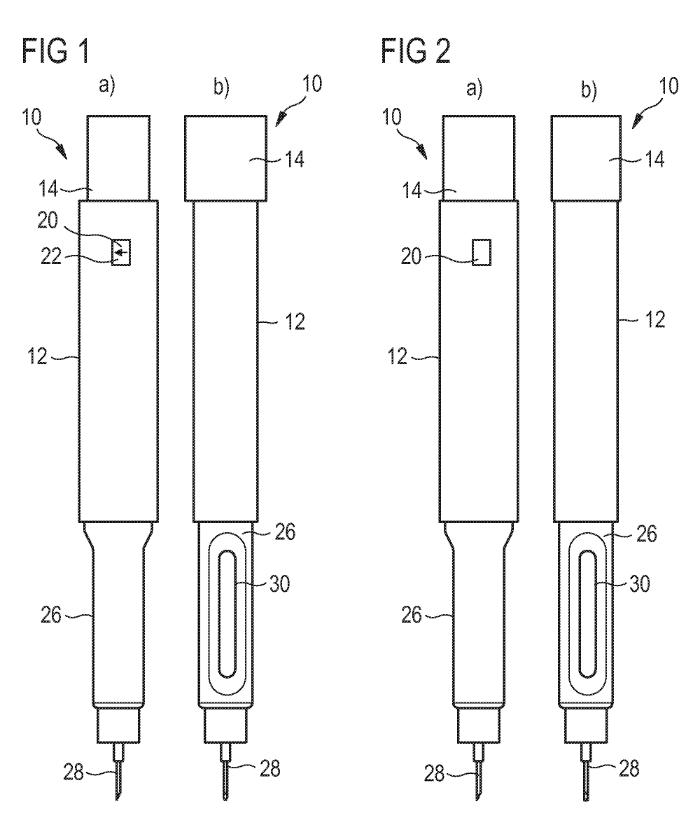

FIGS. 1a and 1b show a side view of a drug delivery device in a pre-ready state,

FIGS. 2a and 2b show a side view of a drug delivery device while the drug delivery device is in a transient state,

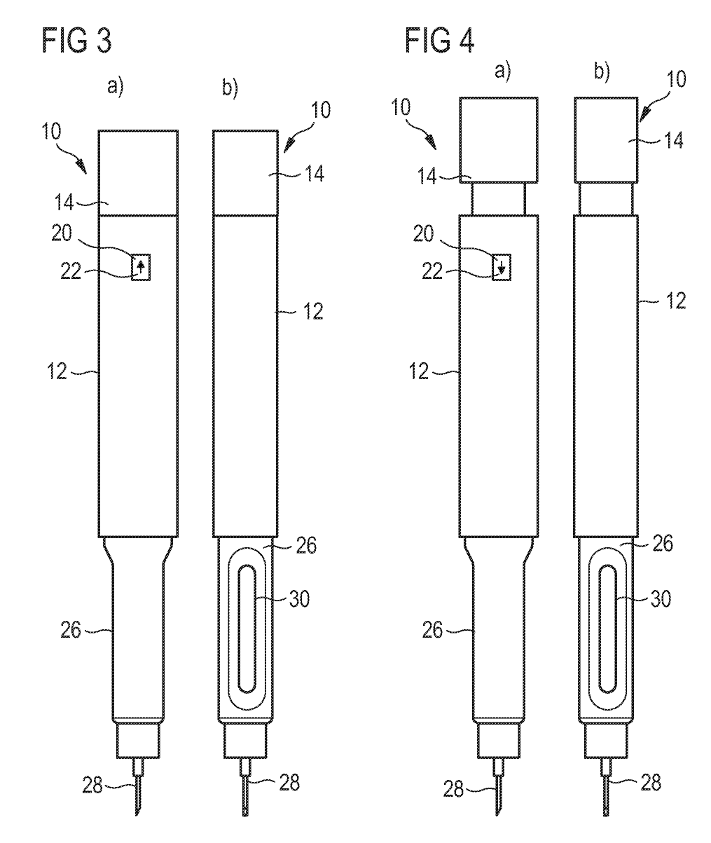

FIGS. 3a and 3b show a side view of a drug delivery device in a ready state when a dose of a medicinal product can be set,

FIGS. 4a and 4b show a side view of a drug delivery device in a ready state when a dose of a medicinal product is set and may be dispensed,

FIGS. 5a, 5b and 5c show the three different states of a pattern located at the button member and the body which forms a cross,

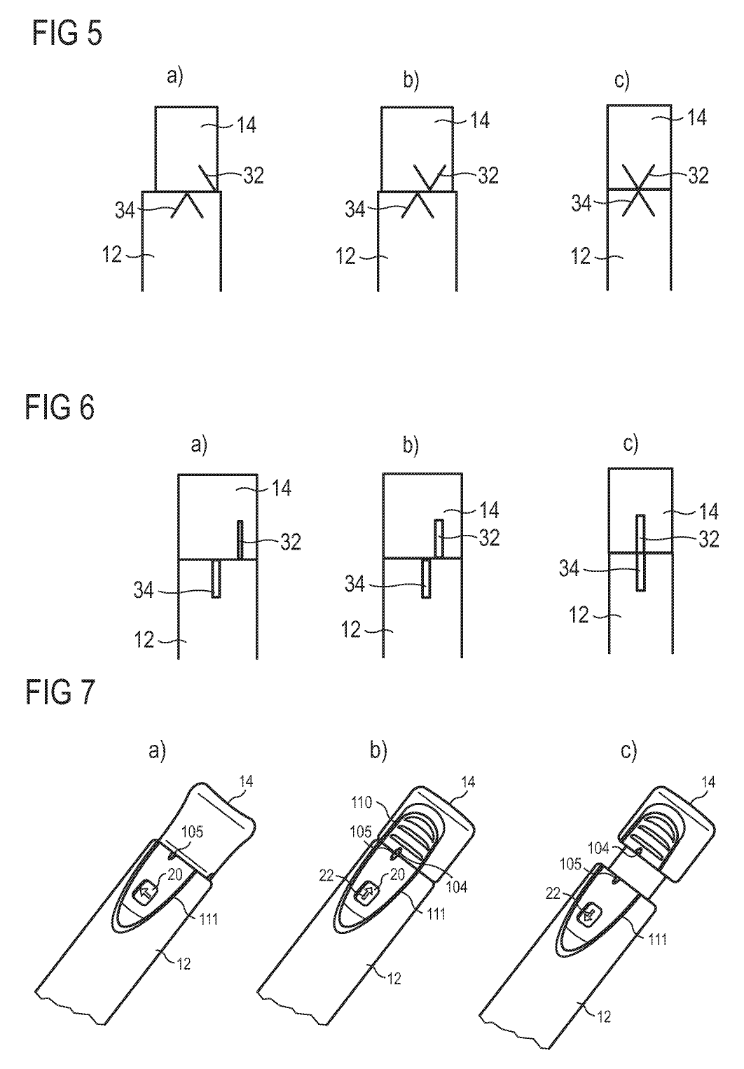

FIGS. 6a, 6b and 6c show the three different states of a haptic feature located at the button member and the body, and

FIGS. 7a, 7b and 7c illustrate a perspective view of three different states of an embodiment according to the present invention.

FIGS. 1 to 4 are shown in two side views, wherein the side view of the drug delivery device shown in FIGS. 1b, 2b, 3b and 4b is 90° rotated relative to the side view of the drug delivery device shown in the FIGS. 1a, 2a, 3a and 4a.

Some embodiments of the drug delivery device according to the present disclosure will now be discussed with reference to FIG. 1-7. Identical reference numerals denote identical or comparable components.

FIGS. 1a and 1b show a side view of a pen-type drug delivery device in a pre-ready state. The side view shown in FIG. 1b is 90° rotated relative to the side view shown in the FIG. 1a.

The drug delivery device 10 shown in FIG. 1a comprises a proximal end, at which a button member 14 is located. A needle unit 28 is located at a distal end of the drug delivery device. Through the needle unit 28 a medicinal product may be dispensed out of a medicament cartridge, which is not explicitly shown.

The drug delivery device 10 comprises a body 12 and a cartridge holder 26, inside which the medicament cartridge is arranged. Body 12 has a first window 20 where an indicator 22 can be seen. The indicator 22 is located on a drive sleeve which is covered by the body 12 except in the region of the window 20 and therefore is not illustrated.

Indicator 22 is represented by an arrow which points in a left direction. The arrow indicates that the user has to twist the button member 14 in the indicated direction relative to the body 12. Such twisting action will prepare the drug delivery device 10.

The fact that the drug delivery device 10 is in its pre-ready state can be seen by means of a misalignment between the button member 14 and the body 12 of the drug delivery device 10.

The misalignment can be perceived visually by the user for example through an oval cross-sectional area of both the body 12 and the button member 14 which are arranged at 90° to each other in the pre-ready state. This misalignment is also perceivable by touching the button member 14 and the body 12. The user can feel the misalignment between the body 12 and the button member 14.

Additionally or alternatively, a sensor, that is not explicitly shown, may detect the misalignment and provide a signal that indicates that the drug delivery device 10 is in the pre-ready state and that the user has to prepare the drug delivery device 10.

A way to indicate the pre-ready state can also be by having a pattern on the body 12 and on the button member 14. This pattern is not complete while the body 12 and the button member 14 are not aligned.

The pattern might be a picture with one part of the picture on the body 12 and the other part on the button member 14. In the pre-ready state, both parts are not completing each other.

The pattern can also be haptically perceivable. For example, a simple protrusion can be located along the longitudinal axis of the drug delivery device 10. One protrusion can be located at the button member 14 and one protrusion can be located at the body 12. In the pre-ready state, both parts are not aligned along the longitudinal axis of the drug delivery device 10.

All possible patterns to make the misalignment of the body 12 and the button member 14 perceivable to the user of the drug delivery device 10 can be applied to the surface of the button member 14 and the body 12 or can be integrally formed with the body 12 and the button member 14.

In FIG. 1b, in a 90° rotated view of FIG. 1a it is shown that the misalignment between the button member 14 and the body 12 is obvious from every direction the user may look onto the drug delivery device 10.

The cartridge holder 26 comprises a window 30 through which a medicament cartridge, which is not explicitly shown and which comprises a medicinal product, can be seen.

FIGS. 2a and 2b show a side view of a pen-type drug delivery device 10 in a transient state in which the drug delivery device 10 is partially prepared. The side view shown in FIG. 2b is 90° rotated relative to the side view shown in the FIG. 2a.

The drug delivery device 10 comprises a button member 14, a body 12, a cartridge holder 26, a first window 20 at the body 12 and a second window 30 at the cartridge holder 26. A needle unit 28 is shown at the distal end of the drug delivery device 10.

Instead of a window as shown, a transparent material for the body can be used. Body 12 may comprise a transparent material in the area, where window 30 is located.

Through the first window 20 which is located at the body 12, a blank region on the drive sleeve is shown. The drive sleeve is not explicitly shown because it is totally covered by the body 12 except for the region of window 20.

The drug delivery device 10 has a proximal end, where a button member 14 is located and a distal end, where a needle unit 28 is located through which a medicinal product may be dispensed out of an assembled medicament cartridge, which is not explicitly shown.

The fact that the drug delivery device 10 is in its transient state can be seen by means of an obvious misalignment between the button member 14 and the body 12 of the drug delivery device 10.

The misalignment can be perceived visually by the user, for example through an oval cross-sectional area of both the body 12 and the button member 14 which are arranged at 90° to each other in the pre-ready state. This misalignment is also perceivable by touching the button member 14 and the body 12. Additionally or alternatively, a sensor, that is not explicitly shown, may detect the misalignment and provide a signal that indicates that the drug delivery 10 is in its transient state.

A way to indicate the transient state can be to have a pattern on the body 12 and on the button member 14. This pattern is not fitting together while the body 12 and the button member 14 are not aligned.

The pattern might be a picture with one part of the picture on the body 12 and the other part on the button member 14. In the transient state, both parts are not completing each other.

The pattern can also be haptically perceivable. For example, a simple protrusion can be located along the longitudinal axis of the drug delivery device 10. One protrusion can be located at the button member 14 and one protrusion can be located at the body 12. In the transient state, both parts are not aligned along the longitudinal axis of the drug delivery device 10.

All possible patterns to make the misalignment of the body 12 and the button member 14 perceivable to the user of the drug delivery device 10 can be applied to the surface of the button member 14 and the body 12 or can be integrally formed with the body 12 and the button member 14.

During the transient state, the user continues to twist the button member 14 relative to the body 12 of the drug delivery device 10. The twisting reduces the misalignment of the button member 14 relative to the body 12. The blank space on the drive sleeve indicates the user to keep on twisting, until a new indicator can be seen through the first window 20. Alternatively a series of arrows could appear in window 20 as the drive sleeve is rotated through the transient state.

At the end of the transient state, a clamp or retainer (not illustrated here) snaps in and holds the button member in the ready state. As such the button member is kept irreversibly in the ready state. For such purpose, the button member or the drive assembly may comprise a retainer element, which at the end of the twisting operation fits into a recess or something similar thereby fixating the button member in the ready state. Alternatively, the button member may comprise a guiding track on its internal surface, which fits into a protrusion on the drive assembly or housing of the drug delivery device. During the twisting operation, the protrusion forces the button member to a specific motion, pre-defined by the guiding path. A retainer element is arranged at the end of the guiding path, which snaps in as soon as the protrusion has glided over the element, thereby preventing the button member from being twisted backwards.

Such element allows an uni-directional coupling at the end of the twisting motion and the transient state.

FIGS. 3a and 3b show a side view of a pen-type drug delivery device 10 in a prepared state of the drug delivery device 10. The side view shown in FIG. 3b is 90° rotated relative to the side view shown in the FIG. 3a. In the ready state, the drug delivery device 10 is ready for setting and dispensing a dose of a medicinal product.

The drug delivery device 10 comprises a button member 14, a body 12, a cartridge holder 26, a first window 20 at the body and a second window 30 at the cartridge holder 26. A needle unit 28 is shown at the distal end of the drug delivery device 10. Through the first window 20 which is located at the body 12, an indicator 22 located on the drive sleeve is shown. The drive sleeve is covered by the body 12 except for the region of the window 20.

The drug delivery device 10 has a proximal end, where a button member 14 is located and a distal end, where a needle unit 28 is located through which a medicinal product may be dispensed out of a medicament cartridge which is not explicitly shown.

The indicator 22 is represented by an arrow which is pointing towards the proximal end of the drug delivery device 10. The indicator 22 indicates to the user that the next possible movement of the button member 14 relative to the body 12 is to be pulled in proximal direction. For further use of the drug delivery device, the user has to pull the button member in proximal direction.

The fact that the drug delivery device 10 is in its ready state can be seen by means of the alignment between the button member 14 and the body 12 of the drug delivery device 10.

The alignment can be perceived visually by the user for example through an oval cross-sectional area of both the body 12 and the button member 14. Both oval cross sections are aligned. The alignment is also perceivable by touching the button member 14 and the body 12. Additionally or alternatively, a sensor, that is not explicitly shown, may detect the alignment and provide a signal that indicates the user that the drug delivery device 10 is in the ready state.

A way to indicate the ready state can be, to have a pattern on the body 12 and on the button member 14. This pattern fits together, completes or matches when the body 12 and the button member 14 are aligned.

The pattern might be a picture with one part of the picture on the body 12 and the other part on the button member 14. In the ready state, both parts are completing each other.

The pattern can also be haptically perceivable. For example, a simple protrusion can be located along the longitudinal axis of the drug delivery device 10. One protrusion can be located at the button member 14 and one protrusion can be located at the body 12. In the ready state, both parts are aligned along the longitudinal axis of the drug delivery device 10.

All possible patterns to make the alignment of the body 12 and the button member 14 perceivable to the user of the drug delivery device 10 can be applied to the surface of the button member 14 and the body 12 or can be integrally formed with the body 12 and the button member 14.

FIGS. 4a and 4b show a side view of a pen-type drug delivery device 10 in a ready state of the drug delivery device 10 wherein a dose of a medicinal product is set. The side view shown in FIG. 4b is 90° rotated relative to the side view shown in the FIG. 4a.

The drug delivery device 10 comprises a button member 14, a body 12, a cartridge holder 26, a first window 20 at the body 12 and a second window 30 at the cartridge holder 26. A needle unit 28 is shown at the distal end of the drug delivery device 10. Through the first window 20 which is located at the body 12, an indicator 22 located on the drive sleeve is shown. The drive sleeve is covered by the body 12 except for the window region 20.

The drug delivery device 10 has a proximal end, where a button member 14 is located and a distal end, where a needle unit 28 is located through which a medicinal product may be dispensed out of a medicament cartridge which is not explicitly shown.

The indicator 22 is an arrow which points towards the distal end of the drug delivery device 10. The body 12 and the button member 14 are aligned and a dose of a medicinal product is set.

The indicator 22 indicates to the user that the next possible movement of the button member 14 relative to the body 12 is to be pushed in distal direction.

FIG. 5a shows a side view of a pen-type drug delivery device comprising a button member 14 and a body 12 in a pre-ready state, wherein a first part 32 of a pattern is located at a distal part of the button member 14 and a second part 34 of the pattern is located at an proximal part of the body 12. The misalignment between the first part 32 and the second part 34 of the pattern and between the body 12 and the button member 14 is visible to the user.

FIG. 5b shows a side view of a pen-type drug delivery device comprising a button member and a body during a transient state, wherein a first part 32 of the pattern is located at the button member 14 and a second part 34 of the pattern is located at the body 12. The misalignment between the first part 32 and the second part 34 of the pattern and between the button member 14 and the body 12 is visible to the user. While further twisting the button member 14 relative to the body 12, the misalignment decreases, which is perceivable by the user.

FIG. 5c shows a side view of a pen-type drug delivery device comprising a button member and a body in a ready state, wherein a first part 32 of the pattern is located at the button member 14 and a second part 34 of the pattern is located at the body 12. The alignment between the first part 32 and the second part 34 of the pattern is visible to the user. The now complete pattern 32, 34 forms a cross which reaches from the button member 14 to the body 12 of the drug delivery device.

In other words the body 12 and the button member 14 are aligned and the pattern is complete. This indicates to the user that the drug delivery device is ready for use and that a dose of a medicinal product can be set and dispensed out of the assembled medicament cartridge.

FIG. 6a shows a side view of a pen-type drug delivery device comprising a button member 14 and a body 12 in a pre-ready state, wherein a first part 32 of a tactile pattern is located at a distal part of the button member 14 and a second part 34 of the tactile pattern is located at an proximal part of the body 12. The misalignment between the first part 32 and the second part 34 of the pattern is both visible and evident by tactile means for the user.

FIG. 6b shows a side view of a pen-type drug delivery device comprising a button member and a body in a transient state, wherein a first part 32 of the tactile pattern is located at the button member 14 and a second part 34 of the tactile pattern is located at the body 12. The misalignment between the first part 32 and the second part 34 of the tactile pattern is both visible and evident by tactile means for the user. With further twisting of the button member 14 relative to the body 12, the misalignment decreases, which is perceivable for the user.

FIG. 6c shows a side view of a pen-type drug delivery device comprising a button member and a body in a ready state, wherein a first part 32 of the tactile pattern is located at the button member 14 and a second part 34 of the tactile pattern is located at the body 12. The alignment between the first part 32 and the second part 34 of the pattern is both visible and evident by tactile means for the user. The now complete pattern 32, 34 forms a line which extends from the button member 14 to the body 12 of the drug delivery device.

In other words both parts form a complete pattern. This indicates to the user, that the drug delivery device is ready for use and that a dose of a medicinal product can be set and dispensed out of an assembled medicament cartridge.

FIGS. 7a) to 7c) illustrates a perspective view of an embodiment combining visual and tactile features to indicate whether the devise is ready for use or still has to be prepared. The device comprises a body 12 with a half-oval recess 111 at its distal end. A window 20 is arranged within recess 111, showing an arrow arranged on an internally arranged drive assembly. The arrow indicates the direction for moving the button 14. Recess 111 also comprises an indicator 105 and some tactile element within the recess for a better gripping.

Button 14 comprises a recess 110 corresponding to the recess 111 of body 12 and a indicator portion 105. In FIG. 7a) which shows a non-prepared state of the device, both recesses are misaligned to each other. Accordingly the indicator portions 105 and 104, respectively do not align as well. During preparation, button 14 is twisted and the recesses 111 and 110 become slowly aligned. At the end of the preparation step as indicated in FIG. 7b), the respective indicator portions 105, 104 and the recesses 111, 110 are aligned. The arrow 22 within window or aperture 20 indicates the new direction, in which button 14 can be moved by a user. FIG. 7c) finally illustrates the state, in which the device has been set up for delivering a fluid. Arrow 22 pointing towards proximal end of the device indicates a push movement for button 14.

The present examples and embodiments are to be considered as illustrative and not restrictive, and the invention is not to be limited to the details given herein, but may be modified within the scope and equivalence of the appended claims.

### REFERENCE NUMERALS

10 drug delivery device
12 body
14 button member
20 first window
22 indicators
26 cartridge holder
28 needle unit
30 second window
32 first part of the pattern
34 second part of the pattern

1. A drug delivery device comprising:
a body and a moveable button member to operate the drug delivery device in a pre-ready state to prepare the drug delivery device and in a subsequent ready state to set and dispense a medicinal product;
wherein in a pre-ready state of the drug delivery device there exists a misalignment between the body and the button member which is perceivable by a user of the drug delivery device and
wherein in a ready state of the drug delivery device, the button member and the body are aligned and the alignment is perceivable by a user of the drug delivery device.

2. A drug delivery device according to claim 1, wherein in the pre-ready state the button member is rotatable with respect to the body.

3. A drug delivery device according to claim 1, wherein in the ready state the button member is axially displaceable with respect to the body.

4. A drug delivery device according to claim 1, wherein in a transient state the drug delivery device is changed from the pre-ready state to the ready state and after having reached the ready state the drug delivery device is irreversibly kept in the ready state.

5. A drug delivery device according to claim 1, wherein at least one of the pre-ready state, the transient state or the ready state of the drug delivery device is audibly, tactile or visibly perceivable by a user of the drug delivery device.

6. A drug delivery device according to claim 1, wherein a cross section along a lateral axis of the body and of the button member is non-circularly constructed.

7. A drug delivery device according to claim 1, wherein in the pre-ready state the body and the button member are rotationally twisted relative to each other.

8. A drug delivery device according to claim 1, wherein a drive sleeve which is located inside the body is operationally coupled to the button member to prepare the drug delivery device in response to a preparing movement of the button member.

9. A drug delivery device according to claim 8, wherein the drive sleeve comprises at least one indicator on its surface that indicates at least one of the pre-ready state, the transient state or the ready state.

10. A drug delivery device according claim 9, wherein the body has a window through which at least one of the at least one indicator on the drive sleeve is visible.

11. A drug delivery device according to claim 1, wherein in the pre-ready state, a misalignment of a pattern shown on both the body and the button member is detectable and in the ready state of the drug delivery device, an alignment of the pattern on the body and the button member is detectable.

12. A drug delivery device according to claim 11, wherein the pattern on the body and on the button member is at least one of
at least one haptic feature;
at least one coloured line;
at least one symbol;
at least one haptic symbol;
at least one coloured symbol.

13. A drug delivery device according to claim 1, wherein the drug delivery device comprises a sensor to detect at least one of the pre-ready state, the transient state or the ready state.

14. A drug delivery device according to claim 13, wherein a display indicates visibly to the user that the drug delivery device has reached the ready state or is in the ready state.

15. A drug delivery device according to claim 1, wherein in the ready state the button member is axially displaceable to set and dispense a dose of a medicinal product.
